(19) United States
(12) Patent Application Publication
(10) Pub. No.: US 2006/0134214 A1
(43) Pub. Date: Jun. 22, 2006

(54) Enteric coated bead comprising epothilone or epothilone analog, and preparation and administration thereof

(76) Inventors: Ismat Ullah, Cranbury, NJ (US); Gary James Wiley, Jackson, NJ (US)

Correspondence Address: LOUIS J. WILLE;BRISTOL-MYERS SQUIBB COMPANY, PATENT DEPARTMENT, P O BOX 4000, PRINCETON, NJ, 08543-4000, US

(21) Appl. No.: 11/281,854

(22) Filed: Nov. 17, 2005

Related U.S. Application Data
(60) Provisional application No. 60/628,968, filed on Nov. 18, 2004.

(51) Int. Cl. A61K 9/24; A61K 31/427

(52) U.S. Cl. 424/472; 514/365

(57) ABSTRACT

Disclosed is an enteric coated bead comprising at least one epothilone or epothilone analog; and a capsule comprising a multitude of the enteric coated beads. Also disclosed are a method of preparing the enteric coated bead and a method of treating cancer or other proliferative diseases using the enteric coated bead.

### CROSS-REFERENCE TO RELATED APPLICATIONS

The present application claims the priority benefit of U.S. Provisional Application No. 60/628,968, filed Nov. 18, 2004, which is incorporated herein by reference in its entirety.

### FIELD OF THE INVENTION

The present invention generally relates to an enteric coated bead comprising at least one epothilone or epothilone analog. A method is provided for preparing the enteric coated bead. Also, a method of treating cancer or other proliferative diseases using the enteric coated bead is provided.

### BACKGROUND OF THE INVENTION

Epothilones are macrolide compounds having utility in the pharmaceutical field. For example, Epothilones A and B are naturally-occurring compounds that can be isolated from certain microorganisms, having the structures:

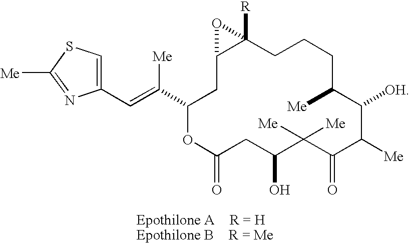

Known epothilones exert microtubule-stabilizing effects similar to TAXOL® and therefore exhibit cytotoxic activity against rapidly proliferating cells, such as occur in cancer and other hyperproliferative cellular diseases (See Angew. Chem. Int. Ed. Engl., Vol. 35, No. 13/14, 1996 and D. M. Bollag, Exp. Opin. Invest. Drugs, 6(7): 867-873, 1997).

Before these epothilones can be used to treat diseases in patients, however, they must be formulated into pharmaceutical compositions that can be administered to the patient; for example, into a dosage form suitable for oral, mucosal (e.g., nasal, sublingual, vaginal, buccal, or rectal), parenteral (e.g., subcutaneous, intravenous, bolus injection, intramuscular, or intraarterial), or transdermal administration. Formulations for oral administration are particularly preferred since they are more convenient and easier to administer than other formulations. Also, the oral route of administration avoids the pain and discomfort of parenteral administration. Oral administration is preferred by patients and results in better patient compliance with dosing schedules.

Oral administration involves passage of the epothilone compound through the stomach, where it is exposed to low pH gastric fluids, and then passage into the small intestine, where the epothilone compound is absorbed into the bloodstream. Transit time through the stomach is approximately two hours. The pH of the stomach is approximately 1 to 3. The small intestine, which includes the duodenum, jejunum, and the ileum, has pH values for these regions of approximately 5 to approximately 7.2. However, epothilone compounds, such as epothilones and epothilone analogs, may undergo degradation, decomposition, or deactivation in aqueous solution, particularly in acidic solutions. In oral administration, the bioavailability of the epothilone compound is dependent upon minimizing loss of the epothilone compound in the acid conditions encountered during passage through the stomach.

U.S. Pat. No. 6,576,651 discloses a method for oral administration of epothilone compounds. The method comprises orally administering the epothilone compound, and orally administering one or more pharmaceutically acceptable acid neutralizing buffers. The acid neutralizing buffer may be administered prior to, concurrently, or after the administration of the epothilone compound. The disclosed method allows the delivery of the epothilone compound to a mammal while reducing or avoiding the degradation, decomposition, or deactivation of the epothilone compound by the gastrointestinal system, particularly by gastric fluid in the stomach. However, raising the pH of the stomach can cause stomach upset and indigestion. Further, oncology patients often need to take other medicines, for some of which alkaline stomach conditions may not be desirable. Desired are an oral dosage form and a method for the oral administration of an epothilone compound that do not require neutralization of the stomach acid.

The epothilone compounds typically require special handling during the manufacture of the dosage form. For example, manufacturing equipment may require special engineering controls to reduce or remediate dust formed during milling to reduce the particle size of the epothilone compounds. Further, desired is a dosage form that may be prepared by a method that minimizes dusting of the epothilones compounds.

In accordance with the present invention, an enteric coated bead comprising at least one epothilone or epothilone analog is provided. The enteric coated bead is suitable for oral administration of the at least one epothilone or epothilone analog without requiring the use of an acid neutralizing buffer. Further, the enteric coated bead may be prepared by a method that reduces or eliminates dusting of the epothilone or epothilone analog.

### SUMMARY OF THE INVENTION

The present invention relates to an enteric coated bead comprising: a coated particle comprising a base particle and an active ingredient layer disposed on the base particle, wherein the active ingredient layer comprises at least one epothilone or epothilone analog, or a pharmaceutically acceptable salt, solvate, clathrate, hydrate, or prodrug thereof, and at least one binder; and an enteric coating encapsulating the coated particle.

Also provided are a process for preparing the enteric coated bead, a capsule comprising the enteric coated beads, and a method of treating cancer or other proliferative diseases comprising orally administering the enteric coated bead to a patient in need thereof.

### DETAILED DESCRIPTION OF THE INVENTION

### Definitions

The following are definitions of various terms used herein to describe the present invention. These definitions apply to the terms as they are used throughout this specification, unless otherwise limited in specific instances, either individually or as part of a larger group.

The term “alkyl” refers to optionally substituted straight- or branched-chain saturated hydrocarbon groups having from 1 to about 20 carbon atoms, preferably from 1 to about 7 carbon atoms. The expression “lower alkyl” refers to alkyl groups having from 1 to 4 carbon atoms. A “substituted lower alkyl” refers to an alkyl group having from 1 to 4 carbon atoms and one, two, or three (preferably one or two) substituents selected from those recited for “substituted alkyl” groups.

The term “substituted alkyl” refers to an alkyl group substituted by, for example, one to four substituents (preferably one to two substituents), such as, halo, trifluoromethyl, trifluoromethoxy, hydroxy, alkoxy, cycloalkyoxy, heterocyclooxy, oxo (═O), alkanoyl, aryl, aryloxy, aralkyl, alkanoyloxy, amino, alkylamino, arylamino, aralkylamino, cycloalkylamino, heterocycloamino, disubstituted amino (in which the two substituents on the amino group are selected from alkyl, aryl, and aralkyl), alkanoylamino, aroylamino, aralkanoylamino, substituted alkanoylamino, substituted arylamino, substituted aralkanoylamino, thiol, alkylthio, arylthio, aralkylthio, cycloalkylthio, heterocyclothio, alkylthiono, arylthiono, aralkylthiono, alkylsulfonyl, arylsulfonyl, aralkylsulfonyl, sulfonamido (e.g., —SO₂NH₂), substituted sulfonamido, nitro, cyano, carboxy, carbamyl (e.g., —C(═O)NH₂), substituted carbamyl (e.g., —C(═O)NRR′, wherein R and R′ are selected from hydrogen, alkyl, and aryl, provided at least one of R and R′ is other than hydrogen), alkoxycarbonyl, aryl, substituted aryl, guanidino, and heterocyclo, such as indolyl, imidazolyl, furyl, thienyl, thiazolyl, pyrrolidyl, pyridyl, pyrimidyl, and the like. Wherein, as noted above, the substituents themselves are further substituted, such further substituents are selected from halogen, alkyl, alkoxy, aryl, and aralkyl. The definitions given herein for alkyl and substituted alkyl apply as well to the alkyl portion of alkoxy groups.

The term “halogen” or “halo” refers to fluorine, chlorine, bromine, and iodine.

The term “aryl” refers to an optionally substituted monocyclic or bicyclic aromatic hydrocarbon group having from about 6 to about 12 carbon atoms in the ring portion, for example, phenyl and naphthyl.

The term “aralkyl” refers to an aryl group bonded to a larger entity through an alkyl group, for example, a benzyl group.

The term “substituted aryl” refers to an aryl group substituted by, for example, one to four substituents (preferably one to two substituents) such as alkyl, substituted alkyl, halo, trifluoromethyl, trifluoromethoxy, hydroxy, alkoxy, cycloalkyloxy, heterocyclooxy, alkanoyl, alkanoyloxy, amino, alkylamino, dialkylamino, aralkylamino, cycloalkylamino, heterocycloamino, alkanoylamino, thiol, alkylthio, cycloalkylthio, heterocyclothio, ureido, nitro, cyano, carboxy, carboxyalkyl, carbamyl, alkoxycarbonyl, alkylthiono, arylthiono, alkysulfonyl, sulfonamido, aryloxy, and the like. The substituent may be further substituted by one or more members selected from halo, hydroxy, alkyl, alkoxy, aryl, substituted alkyl, substituted aryl, and aralkyl.

The term “cycloalkyl” refers to optionally substituted saturated cyclic hydrocarbon ring systems, preferably containing 1 to 3 rings and 3 to 7 carbons per ring, which may be further fused with an unsaturated C₃-C₇ carbocyclic ring. Exemplary groups include cyclopropyl, cyclobutyl, cyclopentyl, cyclohexyl, cycloheptyl, cyclooctyl, cyclodecyl, cyclododecyl, and adamantyl. Exemplary substituents include one or more alkyl or substituted alkyl groups as described above, or one or more of the groups described above as substituents for alkyl groups. Additionally, a cycloalkyl may contain a carbon-carbon bridge of one to two bridgehead carbon atoms, and/or one or two (preferably one) of the ring carbon atoms optionally may be replaced with a carbonyl group (substituted with keto).

The terms “heterocycle”, “heterocyclic” and “heterocyclo” refer to an optionally substituted, unsaturated, partially saturated, or fully saturated, aromatic or nonaromatic cyclic group, for example, which is a 4- to 7-membered monocyclic, 7- to 11-membered bicyclic, or 10- to 15-membered tricyclic ring system, which has at least one heteroatom in at least one carbon atom-containing ring. Each ring of the heterocyclic group containing a heteroatom may have 1, 2 or 3 heteroatoms selected from nitrogen atoms, oxygen atoms, and sulfur atoms, where the nitrogen and sulfur heteroatoms may also optionally be oxidized and the nitrogen heteroatoms may also optionally be quaternized. The heterocyclic group may be attached at any heteroatom or carbon atom.

Exemplary monocyclic heterocyclic groups include pyrrolidinyl, pyrrolyl, pyrazolyl, oxetanyl, pyrazolinyl, imidazolyl, imidazolinyl, imidazolidinyl, oxazolyl, oxazolidinyl, isoxazolinyl, isoxazolyl, thiazolyl, thiadiazolyl, thiazolidinyl, isothiazolyl, isothiazolidinyl, furyl, tetrahydrofuryl, thienyl, oxadiazolyl, piperidinyl, piperazinyl, 2-oxopiperazinyl, 2-oxopiperidinyl, 2-oxopyrrolidinyl, 2-oxazepinyl, azepinyl, 4-piperidonyl, pyridyl, N-oxo-pyridyl, pyrazinyl, pyrimidinyl, pyridazinyl, tetrahydropyranyl, tetrahydrothiopyranyl, tetrahydrothiopyranyl sulfone, morpholinyl, thiomorpholinyl, thiomorpholinyl sulfoxide, thiomorpholinyl sulfone, 1,3-dioxolane, and tetrahydro-1,1-dioxothienyl, dioxanyl, isothiazolidinyl, thietanyl, thiiranyl, triazinyl, triazolyl, and the like.

Exemplary bicyclic heterocyclic groups include benzothiazolyl, benzoxazolyl, benzothienyl, quinuclidinyl, quinolinyl, quinolinyl-N-oxide, tetrahydroisoquinolinyl, indolyl, isoquinolinyl, benzimidazolyl, benzopyranyl, indolizinyl, benzofuryl, chromonyl, coumarinyl, cinnolinyl, quinoxalinyl, indazolyl, pyrrolopyridyl, furopyridinyl (such as furo[2,3-c]pyridinyl, furo[3,1-b]pyridinyl], or furo[2,3-b]pyridinyl), dihydroisoindolyl, dihydroquinazolinyl (such as 3,4-dihydro-4-oxo-quinazolinyl), benzisothiazolyl, benzisoxazolyl, benzodiazinyl, benzofurazanyl, benzothiopyranyl, benzotriazolyl, benzpyrazolyl, dihydrobenzofuryl, dihydrobenzothienyl, dihydrobenzothiopyranyl, dihydrobenzothiopyranyl sulfone, dihydrobenzopyranyl, indolinyl, isochromanyl, isoindolinyl, naphthyridinyl, phthalazinyl, piperonyl, purinyl, pyridopyridyl, quinazolinyl, tetrahydroquinolinyl, thienofuryl, thienopyridyl, thienothienyl, and the like.

Smaller heterocyclos, such as, epoxides, aziridines, and the like, are also included.

Exemplary substituents for the groups “heterocycle,” “heterocyclic,” and “heterocyclo” include alkyl, substituted alkyl, or one or more substituent groups as described above for substituted alkyl or substituted aryl groups.

The term “alkanoyl” refers to —C(═O)-alkyl.

The term “substituted alkanoyl” refers to —C(═O)-substituted alkyl.

The term “heteroatoms” shall include oxygen, sulfur, and nitrogen.

Certain epothilones and epothilone analogs may form salts with a variety of organic and inorganic acids. Such salts include those formed with hydrogen chloride, hydrogen bromide, methanesulfonic acid, hydroxyethanesulfonic acid, sulfuric acid, acetic acid, trifluoroacetic acid, maleic acid, benzenesulfonic acid, toluenesulfonic acid, and various others as are recognized by those of ordinary skill in the art of pharmaceutical compounding. In addition, zwitterions can be formed and are included within the term salts as used herein.

As used herein, the term “epothilone” refers to a compound selected from epothilone A, epothilone B, epothilone C, epothilone D, epothilone E, and epothilone F. Epothilones C to F have the following structures:
I. Epothilone C (R=H) and epothilone D (R=Me) 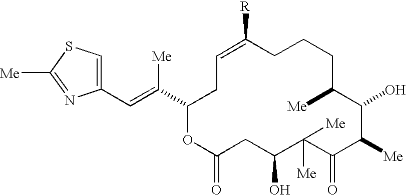
II. Epothilone E (R=H) and epothilone F (R=Me)

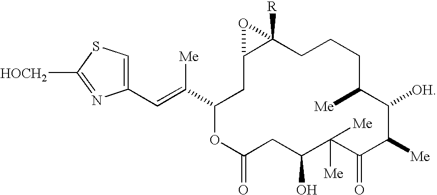

As used herein, the term “epothilone analog” refers to a compound of the formula I: 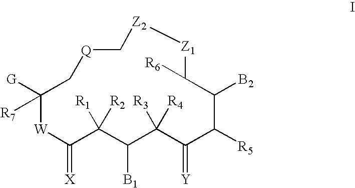
wherein:

Q is

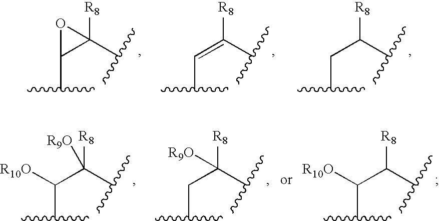

W is O or NR₁₅;

G is alkyl, substituted alkyl, aryl, substituted aryl, heterocyclo, substituted heterocyclo,

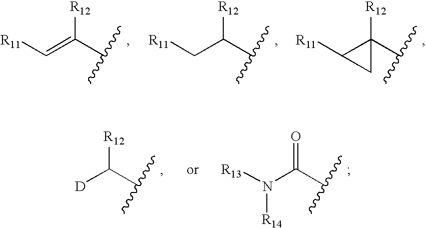

X is O or (H, H);

Y is O, (H, OR₁₆), (OR₁₇, OR₁₇), NOR₁₈, (H, NHOR₁₉), (H, NR₂₀R₂₁), (H, H), or CHR₂₂, wherein (OR₁₇, OR₁₇) can be a cyclic ketal;

Z₁ and Z₂ are independently CH₂, O, NR₂₃, S, and/or SO₂, wherein only one of Z₁ and Z₂ can be a heteroatom;

B₁ and B₂ are independently OR₂₄, OCOR₂₅, and/or O—C(═O)—NR₂₆R₂₇, and when B₁ is OH and Y is (OH, H), they can form a six-membered ring ketal or acetal;

D is NR₂₈R₂₉, NR₃₀COR₃₁, or saturated heterocyclo;

R₁, R₂, R₃, R₄, R₅, R₆, R₇, R₁₃, R₁₄, R₁₈, R₁₉, R₂₀, R₂₁, R₂₂, R₂₆, and R₂₇ are independently H, alkyl, substituted alkyl, and/or aryl, and when R₁ and R₂ are alkyl can be joined to form a cycloalkyl, and when R₃ and R₄ are alkyl can be joined to form a cycloalkyl;

R₉, R₁₀, R₁₆, R₁₇, R₂₄, R₂₅, and R₃₁ are independently H, alkyl, and/or substituted alkyl;

R₈, R₁₁, R₁₂, R₂₈, R₃₀, R₃₂, and R₃₃ are independently H, alkyl, substituted alkyl, aryl, substituted aryl, cycloalkyl, and/or heterocyclo; and

R₁₅, R₂₃, and R₂₉ are independently H, alkyl, substituted alkyl, aryl, substituted aryl, cycloalkyl, heterocyclo, R₃₂C═O, R₃₃SO₂, hydroxy, O-alkyl, and/or O-substituted alkyl;
with the proviso that the term “epothilone analog” expressly excludes epothilone A, epothilone B, epothilone C, epothilone D, epothilone E, epothilone F, and Ixabepilone. Ixabepilone is [1S-[1R*,3R*(E),7R*,10S*,11R*,12R*,16S*]]-7,11-Dihydroxy-8,8,10,12,16-pentamethyl-3-[1-methyl-2-(2-methyl-4-thiazolyl)ethenyl]-4-aza-17-oxabicyclo[14.1.0]heptadecane-5,9-dione and has the structure:

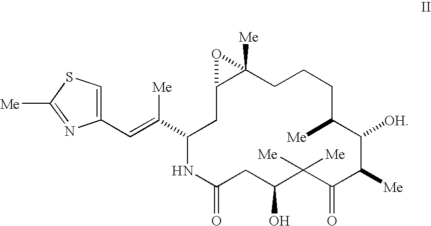

One example of an epothilone analog is 1S-[1R*,3R*(E),7R*,10S*,11R*,12R*,16S*]]-3-[2-[2-(Aminomethyl)-4-thiazolyl]-1-methylethenyl]-7,11-dihydroxy-8,8,10,12,16-pentamethyl-4,17-dioxabicyclo[14.1.0]heptadecane-5,9-dione, which has the structure:

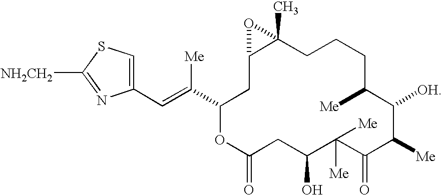

Examples of various epothilones and epothilone analogs, Ixabepilone, and their preparations are disclosed in U.S. Pat. No. 6,262,094 B1; U.S. Pat. No. 6,288,237 B1; U.S. Pat. No. 6,291,684 B1; U.S. Pat. No. 6,316,630 B1; U.S. Pat. No. 6,320,045 B1; U.S. Pat. No. 6,359,140 B1; U.S. Pat. No. 6,369,234 B1; U.S. Pat. No. 6,380,394 B1; U.S. Pat. No. 6,380,395 B1; U.S. Pat. No. 6,399,638 B1; U.S. Pat. No. 6,498,257 B1; U.S. Pat. No. 6,518,421 B1; U.S. Pat. No. 6,593,115 B1; U.S. Pat. No. 6,605,599 B1; U.S. Pat. No. 6,613,912 B1; U.S. Pat. No. 6,624,310 B1; U.S. Pat. No. 6,719,540 B1; U.S. Patent Application Publication 20030087888 A1; U.S. Patent Application Publication 20030186965 A1; U.S. Patent Application Publication 2003/0187039 A1; U.S. Patent Application Publication 2003042217 A1; U.S. Patent Application Publication 20040014982 A1; WO 93/10121; WO 98/22461; WO 99/58534; WO 00/39276; and WO 2004/026254 A2, each of which is incorporate in its entirety, herein, by reference.

As used herein, “substantially free of moisture” refers to a composition comprising less than about 4 weight % water, preferably less than about 3 weight % water, and more preferably, less than about 2 weight % water, based on the weight of the composition. Examples of suitable ranges for “substantially free of moisture” include from zero to about less than about 4 weight %, preferably from zero to about less than about 3 weight %, and more preferably from zero to less than about 2 weight %, based on the weight of the composition.

The present invention relates to an enteric coated bead comprising at least one epothilone or epothilone analog, which is suitable for oral administration to a patient. The enteric coated bead comprises a coated particle, which comprises an active ingredient layer disposed on a base particle; and an enteric coating encapsulating the coated particle. The active ingredient layer comprises at least one epothilone or epothilone analog, and at least one binder. The enteric coating is capable of protecting the at least one epothilone or epothilone analog, which is susceptible to degradation, decomposition, or deactivation during exposure to acidic conditions, from low pH gastric fluids typically encountered during passage through the stomach into the intestine. The enteric coating is capable of minimizing or preventing exposure of the active ingredient layer to stomach acid. This prevents the at least one epothilone or epothilone analog from being released in the stomach or the stomach acid from penetrating through to the active ingredient layer. Upon passage of the enteric coated bead to the small intestine, the enteric coating partially or completely dissolves in the higher pH conditions encountered in the intestine, leading to the release of the epothilone or epothilone analog and its subsequent passage to the bloodstream of the patient.

The enteric coated bead comprises a coated particle encapsulated by an enteric coating. The coated particle comprises a base particle, which provides a seed particle for the application of the active ingredient layer. The base particle comprises a pharmaceutically acceptable material that is capable of carrying the active ingredient layer. Generally, the base particle comprises, for example, a pharmaceutically inert material, such as, for example, sugar, starch, microcrystalline cellulose, lactose, or combinations thereof. Optionally, the base particle may further comprise one or more active agents. The shape of the base particle is typically spherical or semispherical, although other shapes are contemplated. Average diameters for the base particles are typically, for example, in the range of from about 0.1 millimeters to about 5 millimeters. Examples of suitable base particles include Nu-Pareil™ Sugar Spheres NF (Sugar Spheres, NF by Chr. Hansen, Inc., WI) and Celphere™ microcrystalline cellulose spheres (Asahi Kasei Kogyo Kabushiki Kaisha Corp., Japan). Typically, the enteric coated bead comprises, for example, from about 10 to about 80 weight % base particle, preferably from about 15 to about 70 weight % base particle, and more preferably from about 20 to about 65 weight % base particle, based on the weight of the enteric coated bead. Preferably, the base particle is substantially free of moisture. More preferably, the base particle comprises less than 3 weight % water, based on the weight of the base particle.

The coated particle comprises an active ingredient layer disposed on the base particle. The active ingredient layer is applied to the base particle and may form a surface layer on the surface of the base particle, absorb into the base particle, or a combination thereof. The active ingredient layer may be completely or partially distributed on, in, and/or beneath the surface of the base particle. Preferred is an active ingredient layer that is uniformly disposed on the surface of the base particle.

The active ingredient layer comprises at least one epothilone or epothilone analog, or a pharmaceutically acceptable salt, solvate, clathrate, hydrate or prodrug thereof. In addition to the at least one epothilone or epothilone analog, the active ingredient layer may optionally comprise at least one additional active agent, such as an anticancer drug. In one embodiment, two or more different epothilones or epothilone analogs may be included in the active ingredient layer, such as, for example, a mixture of epothilone B and epothilone D. In an alternate embodiment, the active ingredient layer may comprise a mixture of epothilone or epothilone analog, and a pharmaceutically acceptable salt, solvate, clathrate, hydrate or prodrug, such as, for example, a mixture of epothilone F and a clathrate of epothilone F. Suitable levels of the at least one epothilone or epothilone analog, include for example, those in the range of from about 0.1 weight % to about 10 weight %, preferably from about 0.2 weight % to about 5 weight %, and more preferably from about 0.5 weight % to about 4 weight %, based on the weight of the enteric coated bead.

The active ingredient layer also comprises binder. The binder may be employed to improve adhesion of the epothilones or epothilone analog to the base particle and/or to provide cohesion of the active ingredient layer. Materials suitable as binders include, for example, starch; gelatin; sugars such as sucrose, glucose, dextrose, molasses, and lactose; natural and synthetic gums such as acacia, sodium alginate, methyl cellulose, carboxymethylcellulose, and polyvinylpyrrolidone (PVP) polymers and copolymers such as polyvinylpyrrolidone-polyvinyl acetate (PVP-PVA) copolymers; celluloses such as ethylcellulose, hydroxypropyl cellulose, and hydroxypropyl methyl cellulose; polyethylene glycol; and waxes. For example, suitable commercially available materials include Avicel™ PH 101, Avicel™ RC 591, and Avicel™ CL611 cellulose crystallite materials (FMC Corp, PA). One or more different binders may be used in the active ingredient layer. One or more optional ingredients that may be included in the active ingredient layer are, for example, buffers, antifoam agents, and plasticizers. The enteric coated bead may comprise, for example, from about 2 to about 80 weight % of the active ingredient layer, preferably from about 10 to about 70 weight % of the active ingredient layer, and more preferably from about 20 to about 60 weight % of the active ingredient layer, based on the weight of the enteric coated bead. Preferably, the active ingredient layer is substantially free of moisture.

The enteric coated bead has an enteric coating that encapsulates the coated particle. The enteric coating is insoluble or has low solubility in the acid solutions characteristic of the gastric fluids encountered in the stomach, such pH values of less than about 3. At higher pH values, such as those encountered in the small intestine, the enteric coating dissolves to allow the release of the at least one epothilone or epothilone analog. Examples of the higher pH values encountered in the small intestine include pH values of greater than about 4.5, preferably pH values of greater than about 5, and most preferably pH values in the range of from about 5 to about 7.2.

Suitable materials for forming the enteric coating, include, for example, enteric coating polymers, such as, for example, hydroxypropyl methylcellulose phthalate, polyvinyl acetate phthalate, cellulose acetate phthalate, acrylic acid copolymers, and methacrylic acid copolymers. One example of a suitable methacrylic acid copolymer is Eudragit™ L-30-D 55 aqueous copolymer dispersion, which comprises an anionic copolymer derived from methacrylic acid and ethyl acrylate with a ratio of free carboxyl groups to the ethyl ester groups of approximately 1:1, and a mean molecular weight of approximately 250,000, and is supplied as an aqueous dispersion containing 30 weight % solids. Eudragit™ L-30-D 55 aqueous copolymer dispersion is supplied by Rohm-Pharma Co., Germany.

The enteric coated bead may comprise, for example, from about 5 to about 50 weight % of the enteric coating, preferably from about 10 to about 45 weight % of the enteric coating, and more preferably from about 15 to about 40 weight % of the enteric coating, based on the weight of the enteric coated bead. Preferably, the enteric coating is substantially free of moisture.

The enteric coating optionally comprises other materials, such as plasticizers, colorants, antifoam agents, and anti-adherents.

The enteric coated bead optionally comprises one or more subcoat layers that are situated between the base particle and the active ingredient layer, or the active ingredient layer and the enteric coating. The subcoat layer may be employed to minimize contact between the epothilone or epothilone analog contained in the active ingredient layer and an enteric coating comprising acid groups, such as methacrylic acid copolymer. Suitable materials to form the subcoat layer include starch; gelatin; sugars such as sucrose, glucose, dextrose, molasses, and lactose; natural and synthetic gums such as acacia, sodium alginate, methyl cellulose, carboxymethylcellulose, and polyvinylpyrrolidone (PVP) polymers and copolymers such as PVP-PVA copolymers; celluloses such as ethylcellulose, hydroxypropyl cellulose, and hydroxypropyl methyl cellulose; polyethylene glycol; and waxes. The subcoat layer may further comprise one or more plasticizers, such as polyethylene glycol, propylene glycol, triethyl citrate, triacitin, diethyl phthalate, tributyl sebecate, or combinations thereof.

The enteric coated bead optionally comprises other materials such as flavoring agents, preservatives, or coloring agents as may be necessary or desired.

In one non-limiting embodiment, the enteric coated bead is substantially free of moisture. Preferably, the enteric coated bead comprises less than about 4 weight % water, preferably less than about 3 weight % water, and more preferably, less than about 2 weight % water, based on the weight of the enteric coated bead.

The enteric coated bead may be contacted with a hydrophobic material such as talc, magnesium stearate, or fumed silica to form a hydrophobic layer on the surface of the enteric coated bead. The hydrophobic layer is useful to reduce agglomeration of the individual enteric coated beads or to reduce static during the handling of the enteric coated beads.

In one embodiment of the invention, the enteric coated bead comprises: a coated particle and an enteric coating encapsulating the coated particle.

In a second embodiment of the invention, the enteric coated bead comprises: a coated particle comprising a base particle, a subcoat disposed on the base particle, and an active ingredient layer disposed on the subcoat; and an enteric coating encapsulating the coated particle.

In a third embodiment of the invention, the enteric coated bead comprises: a coated particle; a subcoat disposed on the coated particle; and an enteric coating encapsulating the coated particle.

In a fourth embodiment of the invention, the enteric coated bead comprises: a coated particle comprising a base particle, a first subcoat disposed on the base particle, and an active ingredient layer disposed on the subcoat; a second subcoat disposed on the coated particle; and an enteric coating encapsulating the coated particle.

In a fifth embodiment of the invention, the enteric coated bead comprises a coated particle in which the base particle also comprises a second pharmaceutically active ingredient; and an enteric coating encapsulating the coated particle. The enteric coated bead of this embodiment optionally comprises a first subcoat situated between the base particle and the active ingredient layer; and/or a second subcoat situated between the coated particle and the enteric coating. The second pharmaceutically active ingredient may be the epothilone or epothilone analog, or a pharmaceutically acceptable salt, solvate, clathrate, hydrate, or prodrug thereof. Alternatively the second pharmaceutically active ingredient may be another active agent, such as a second anticancer agent.

The enteric coated beads of this invention may be prepared by a process that reduces the exposure of the epothilone or epothilone analog to moisture, heat, or a combination of moisture and heat. Such a process ensures high potency and good uniformity of the active pharmaceutical agent, since the epothilones and the epothilone analogs are susceptible to degradation or decomposition in the presence of water, and especially a combination of moisture and heat.

In one aspect of the present invention, a process is provided for preparing the enteric coated bead, comprising:
a) providing base particles;
b) applying an active ingredient mixture and binder to the base particles, wherein the active ingredient mixture comprises:
   i) at least one epothilone or epothilone analog, or a pharmaceutically acceptable salt, solvate, clathrate, hydrate, or prodrug thereof, and
   ii) solvent, water, or a mixture thereof;
c) drying the base particles having application of the active ingredient mixture to provide coated particles; and
d) applying enteric coating to the coated particles to provide the enteric coated beads.
   In the present process to prepare the enteric coated bead of this invention, the active ingredient mixture may also comprise the binder, thus allowing co-application of a single mixture. Alternatively, the active ingredient mixture and a solution comprising the binder may be premixed immediately prior to application.

The active ingredient mixture comprises at least one epothilone or epothilone analog in solvent, water, or a mixture thereof. The active ingredient mixture may be a solution comprising the epothilone or epothilone analog dissolved in the solvent, water, or mixture thereof. Alternatively, the active ingredient mixture may be an active ingredient suspension comprising particles of the at least one epothilone or epothilone analog dispersed in the solvent, water, or mixture thereof. Suitable solvents include, for example, alcohols such as methanol, ethanol, n-propanol, and isopropanol; and acetone. The active ingredient mixture may be prepared by admixing the at least one epothilone or epothilone analog in solvent, water, or a mixture thereof. Optionally, the binder may be included in the active ingredient mixture. The at least one epothilone or epothilone analog and the optional binder may be combined in any order with the solvent, water, or solvent-water mixture. Typically, mixing is required to minimize any localized concentrations of the at least one epothilone or epothilone analog or the optional binder in the solvent, water, or mixture thereof. Mixing may be provided by a mechanical device, such as a magnetic or overhead stirrer.

In one embodiment, the enteric coated bead of this invention is prepared by applying an active ingredient suspension and binder to the base particles. Preferably, the active ingredient suspension is an aqueous active ingredient suspension comprising the particles of the at least one epothilone or epothilone analog dispersed in an aqueous medium. The aqueous medium comprises greater than about 50 weight % water and optionally, one or more water miscible solvents, based on the weight of the aqueous medium. Preferably the aqueous medium comprises at least about 65 weight % water, more preferably at least about 75 weight % water, and most preferably at least about 95 weight % water, based on the weight of the aqueous medium. The aqueous suspension of the epothilone particles provide a reduction in contact between the aqueous medium and the at least one epothilone or epothilone analog, compared to a solution of the at least one epothilone or epothilone analog, and thus decrease the rate of degradation or decomposition of the at least one epothilone of epothilone analog. The aqueous active ingredient suspension may be prepared by admixing epothilone particles and optionally, the binder, in water and optionally water miscible solvent. The epothilone particles and the optional binder may be combined with the water and/or the optional water miscible solvent in any order. Typically, mixing is required to disperse the epothilone particles and minimize any localized concentrations of the epothilone particles or the optional binder. Suitable size ranges for the epothilone particles in the epothilone dispersion include, for example, less than about 1000 microns, preferably less than 500 microns, and more preferably less than 250 microns. The epothilone particles may be amorphous or crystalline. Preferably, the epothilone particles are crystalline. The active ingredient suspension may comprise from about 1 to about 50 weight % epothilone particles, preferably from about 2 to about 30 weight % epothilone particles, and more preferably from about 3 to about 20 weight % epothilone particles, based on the weight of the epothilone suspension. Preferably, the active ingredient suspension has a pH in the range of from about 6 to about 9, more preferably in the range of from about 6.5 to about 8, and most preferably in the range of from about 6.5 to about 7.5. The active ingredient suspension may optionally comprise other ingredients, such as buffers; dispersing agents such as surfactants or low molecular weight polymers; antifoaming agents, and pH adjusting agents such as acids or bases.

The binder may be provided as a solution or dispersion in water.

In one embodiment, the active ingredient mixture may comprise, for example, from about 1 to about 30 weight % of the at least one binder, preferably from about 2 to about 20 weight % of the at least one binder, and more preferably from about 3 to about 10 weight % of the at least one binder, based on the weight of the epothilone suspension.

The active ingredient mixture and the binder solution may be applied to the base particles as a spray or a stream while the base particles are in motion. The conditions are preferably controlled to minimize particle agglomeration of the base particles. Subsequently, the solvent and/or water is removed from the applied active ingredient mixture leaving the coated particles having the active ingredient layer disposed on the base particle.

The enteric coating may be applied to the coated particles by applying a mixture of the enteric coating as a spray or stream while the coated particles are in motion. The enteric coating mixture may be a solution or a suspension. The conditions are preferably controlled to minimize particle agglomeration. The enteric coating solution comprises the enteric coating material in an aqueous or nonaqueous solvent, or a mixture thereof. Suitable solvents include, for example, alcohols such as methanol or isopropanol; and acetone. Mixtures of solvents or mixtures of water and one or more water miscible solvents may be used. The enteric coating material may be dissolved into the solvent to provide a solution, or alternatively, may be a dispersion of particles, to provide a suspension, such as an aqueous copolymer dispersion. Typically, the enteric coating solution may comprise, for example, from about 5 to about 50 weight % of the enteric coating material, and preferably from about 10 to about 40 weight % of the enteric coating material, based on the weight of the enteric coating solution.

Drying to remove the solvent and/or water may be applied during or after application of the enteric coating solution. In one embodiment, the drying conditions include an inlet drying air temperature in the range of from about 20° C. to about 70° C., an inlet air humidity of less than about 50% relative humidity, a product bed temperature in the range of from about 20° C. to about 40° C., and air flow that is sufficient to remove the free water vapor.

A fluid bed spraying apparatus, a tangential spray coater, or a rotating pan type coater may be employed to spray the active ingredient mixture onto the base particles.

A fluid bed coater is an apparatus that can fluidize particles while simultaneously spraying on and drying a film coat. The fluidizing air is heated to the desired temperature and the air flow adjusted to the flow rate for proper fluidization and drying. A pan coater is an apparatus in which particles are tumbled in a pan while spraying a film coat. Simultaneously air of the proper temperature and airflow passes through the bed of particles to dry the applied film coat.

In one aspect of the invention, a capsule comprising a multitude of the enteric coated beads is provided, suitable for oral administration of the epothilone or epothilone analog. The capsule is prepared by filling a capsule shell, such as a gelatin capsule shell, with the enteric coated beads The capsule allows for easier swallowing during oral administration of the enteric coated beads. Optionally, the capsule comprises at least one hydrophobic material to reduce agglomeration of the individual enteric coated beads in the capsule and/or to reduce static during the loading of the enteric coated beads into the capsule. Generally, the amount of the optional hydrophobic material is preferably kept to a level where it is just enough to prevent particle sticking after the capsule shell has dissolved, but not too much to retard dissolution. Examples of suitable hydrophobic materials include talc, magnesium stearate, stearic acid, glyceryl behenate, hydrogenated cottonseed oil, trimyristin, triplamitan, tristearin, and fumed silica. Examples of commercially available hydrophobic materials include Lubrital™ additive (Penwest Pharmaceutical Co., NJ); Dynasan™ 114, Dynasan™ 116, and Dynasan™ 118 additives (Sasol North America, TX); and Compritol™ 888 ATO additive (Gattefosse Co., France). A preferred hydrophobic material is talc.

### UTILITY

The epothilones and the epothilone analogs are useful as microtubule-stabilizing agents. They are useful in the treatment of a variety of cancers and other proliferative diseases including, but not limited to, the following:
carcinoma, including that of the bladder, breast, colon, kidney, liver, lung, ovary, pancreas, stomach, cervix, thyroid, and skin, including squamous cell carcinoma;
hematopoietic tumors of lymphoid lineage, including leukemia, acute lymphocytic leukemia, acute lymphoblastic leukemia, B-cell lymphoma, T-cell lymphoma, Hodgkins lymphoma, non-Hodgkins lymphoma, hairy cell lymphoma, and Burketts lymphoma;
hematopoietic tumors of myeloid lineage, including acute and chronic myelogenous leukemias and promyelocytic leukemia;
other tumors, including melanoma, seminoma, teratocarcinoma, neuroblastoma, and glioma;
tumors of the central and peripheral nervous system, including astrocytoma, neuroblastoma, glioma, and schwannomas;
tumors of mesenchymal origin, including fibrosarcoma, rhabdomyoscaroma, and osteosarcoma; and
other tumors, including melanoma, xeroderma pigmentosum, keratoacanthoma, seminoma, thyroid follicular cancer, and teratocarcinoma.

The epothilones and the epothilone analogs are useful for treating patients who have been previously treated for cancer, as well as those who have not previously been treated for cancer. The methods and compositions of this invention, including the enteric coated beads, can be used in first-line and second-line cancer treatments. Furthermore, the enteric coated beads are useful for treating refractory or resistant cancers.

The epothilones and the epothilone analogs will also inhibit angiogenesis, thereby affecting the growth of tumors and providing treatment of tumors and tumor-related disorders. Such anti-angiogenesis properties will also be useful in the treatment of other conditions responsive to anti-angiogenesis agents including, but not limited to, certain forms of blindness related to retinal vascularization, arthritis, especially inflammatory arthritis, multiple sclerosis, restinosis, and psoriasis.

The epothilones and the epothilone analogs will induce or inhibit apoptosis, a physiological cell death process critical for normal development and homeostasis. Alterations of apoptotic pathways contribute to the pathogenesis of a variety of human diseases. The subject compounds, as modulators of apoptosis, will be useful in the treatment of a variety of human diseases with aberrations in apoptosis including, but not limited to, cancer and precancerous lesions, immune response related diseases, viral infections, kidney disease, and degenerative diseases of the musculoskeletal system.

The enteric coated beads may also be formulated or co-administered with other therapeutic agents that are selected for their particular usefulness in administering therapies associated with the aforementioned conditions. The enteric coated beads may be formulated with agents to prevent nausea, hypersensitivity, and gastric irritation, such as anti-emetics, and H₁ and H₂ antihistamines. The above therapeutic agents, when employed in combination with the epothilone or epothilone analog, may be used in those amounts indicated in the Physicians' Desk Reference (PDR) or as otherwise determined by one of ordinary skill in the art.

Furthermore, the enteric coated beads may be administered in combination with other anti-cancer and cytotoxic agents and treatments useful in the treatment of cancer or other proliferative diseases. Administration of the enteric coated beads may be prior to, during, or after the administration of the other anti-cancer agents, cytotoxic agents and/or the treatments useful in the treatment of cancer or other proliferative diseases. Especially useful are anti-cancer and cytotoxic drug combinations wherein the second drug chosen acts in a different manner or different phase of the cell cycle, e.g., S phase, than the epothilones and the epothilone analogs, which exert their effects at the G₂-M phase. Examples of classes of anti-cancer and cytotoxic agents include, but are not limited to, alkylating agents, such as nitrogen mustards, alkyl sulfonates, nitrosoureas, ethylenimines, and triazenes; antimetabolites, such as folate antagonists, purine analogues, and pyrimidine analogues; antibiotics, such as anthracyclines, bleomycins, mitomycin, dactinomycin, and plicamycin; enzymes, such as L-asparaginase; farnesyl-protein transferase inhibitors; hormonal agents, such as glucocorticoids, estrogens/antiestrogens, androgens/antiandrogens, progestins, and luteinizing hormone-releasing hormone anatagonists, octreotide acetate; microtubule-disruptor agents, such as ecteinascidins or their analogs and derivatives; microtubule-stabilizing agents such as paclitaxel (TAXOL®), docetaxel (TAXOTERE®); plant-derived products, such as vinca alkaloids, epipodophyllotoxins, and taxanes; topoisomerase inhibitors; prenyl-protein transferase inhibitors; and miscellaneous agents such as, hydroxyurea, procarbazine, mitotane, hexamethylmelamine, platinum coordination complexes such as cisplatin and carboplatin; and other agents used as anti-cancer and cytotoxic agents such as biological response modifiers, growth factors, immune modulators, and monoclonal antibodies. The enteric coated beads may also be used in conjunction with radiation therapy.

Representative examples of these classes of anti-cancer and cytotoxic agents include, but are not limited to, mechlorethamine hydrochlordie, cyclophosphamide, chlorambucil, melphalan, ifosfamide, busulfan, carmustin, lomustine, semustine, streptozocin, thiotepa, dacarbazine, methotrexate, thioguanine, mercaptopurine, fludarabine, pentastatin, cladribin, cytarabine, fluorouracil, doxorubicin (including salts such as doxorubicin hydrochloride), daunorubicin, idarubicin, bleomycin sulfate, mitomycin C, actinomycin D, safracins, saframycins, quinocarcins, discodermolides, vincristine, vinblastine, vinorelbine tartrate, etoposide (including salts such as etoposide phosphate), teniposide, paclitaxel, tamoxifen, estramustine, estramustine phosphate sodium, flutamide, buserelin, leuprolide, pteridines, diyneses, levamisole, aflacon, interferon, interleukins, aldesleukin, filgrastim, sargramostim, rituximab, BCG, tretinoin, irinotecan hydrochloride, betamethosone, capecitabine, gemcitabine hydrochloride, altretamine, and topoteca and analogs or derivatives thereof.

Other examples of these classes of anticancer and cytotoxic agents include, but are not limited to, cisplatin, carboplatin, carminomycin, aminopterin, methotrexate, methopterin, ecteinascidin 743, porfiromycin, 5-fluorouracil (5-FU), 6-mercaptopurine, gemcitabine, cytosine arabinoside, paclitaxel, doxorubicin, daunorubicin, mitomycin C, podophyllotoxin or podophyllotoxin derivatives such as etoposide, etoposide phosphate or teniposide, melphalan, vinblastine, vincristine, leurosidine, vindesine, and leurosine. It is to be understood one or more of the epothilone or epothilone analog compounds may be administered in combination with particular anticancer and cytotoxic agents falling within these classes of agents, for example, the one or more epothilone or epothilone analog may be administered in combination with any 5-FU agents, and/or prodrugs thereof, including without limitation capecitabine (XELODA®).

Further examples of anti-cancer and other cytotoxic agents include the following: cyclin dependent kinase inhibitors as found in WO 99/24416; and prenyl-protein transferase inhibitors as found in WO 97/30992 and WO 98/54966.

Without wishing to be bound to any mechanism or morphology, it is expected that the enteric coated beads, which comprise the at least one epothilone or epothilone analog, could also be used to treat conditions other than cancer or other proliferative diseases. Such conditions include, but are not limited to viral infections such as herpesvirus, poxvirus, Epstein-Barr virus, Sindbis virus, and adenovirus; autoimmune diseases such as systemic lupus erythematosus, immune mediated glomerulonephritis, rheumatoid arthritis, psoriasis, inflammatory bowel diseases, and autoimmune diabetes mellitus; neurodegenerative disorders such as Alzheimer's disease, AIDS-related dementia, Parkinson's disease, amyotrophic lateral sclerosis, retinitis pigmentosa, spinal muscular atrophy, and cerebellar degeneration; AIDS; myelodysplastic syndromes; aplastic anemia; ischemic injury associated myocardial infarctions; stroke and reperfusion injury; restenosis; arrhythmia; atherosclerosis; toxin-induced or alcohol induced liver diseases; hematological diseases such as chronic anemia and aplastic anemia; degenerative diseases of the musculoskeletal system such as osteoporosis and arthritis; aspirin-sensitive rhinosinusitis; cystic fibrosis; multiple sclerosis; kidney diseases; and cancer pain.

The effective amount of the one or more epothilone or epothilone analog may be determined by one of ordinary skill in the art, and includes exemplary dosage amounts for a human for treatment of cancer or other proliferative diseases of from about 1 to 500 mg/m², which may be administered in a single dose or in the form of individual divided doses, such as from 1 to 4 times per day. For example, metastatic breast cancer may be treated by administering a dose of up to 40 mg/m² of the one or more epothilone or epothilone analog once per day every 21 days. It will be understood that the specific dose level and frequency of dosage for any particular subject may be varied and will depend upon a variety of factors including the metabolic stability and length of action of the one or more epothilone or epothilone analog, the species, age, body weight, general health, sex and diet of the subject, the mode and time of administration, rate of excretion, drug combination, and severity of the particular condition.

Preferred subjects for treatment include animals, most preferably mammalian species such as humans, and domestic animals such as dogs, cats and the like, subject to the aforementioned disorders.

Typically the one or more epothilone or epothilone analog is administered until the patient shows a response, for example, a reduction in tumor size, or until dose limiting toxicity is reached. One of ordinary skill in the art will readily know when a patient shows a response or when dose limiting toxicity is reached. The common dose limiting toxicities associated with the one or more epothilone or epothilone analog may include, but are not limited to, fatigue, arthralgia/myalgia, anorexia, hypersensitivity, neutropenia, thrombocytopenia, or neurotoxicity.

One of ordinary skill in the art would readily know how to convert doses from mg/kg to mg/m² given either or both the height and or weight of the patient (See, e.g., http://www.fda.gov/cder/cancer/animalframe.htm).

As discussed above, the enteric coated beads are administered orally. The method of this invention encompasses dosing protocols such as once or twice daily. The oral administration may be daily for a continuous period, weekly, or may be for an intermittent period, such as every 3 to 4 weeks between administration.

In one embodiment, a dosage of from about 1 to about 50 mg/m² is administered daily.

In a different embodiment, a dosage of from about 2 to 150 mg/m² is administered weekly, such as daily for two days followed by 5 days with no oral administration of the enteric coated bead.

In a still different embodiment, a dosage of from about 10 to 300 mg/m² is administered over a period of about 3 to about 4 weeks, such as, for example, daily for one day followed by a period of 20 days with no administration of the enteric coated bead.

### EXAMPLES

The following examples are provided, without any intended limitation, to further illustrate the present invention.

### Example 1

### Preparation of Epothilone Suspension

An active ingredient suspension is prepared containing the Epothilone B. First, 2.783 g Tris powder (tris(hydroxymethyl aminomethane)), 500 ml water, and 1 N HCl are mixed to provide a 0.046 M Tris buffer solution having a pH of 8.1. Next, a mixture of 43.5 g Tris buffer solution (43.5 g) and 2.5 g Opadry™ Clear Coat powder (Colorcon, Inc., PA), as the binder, is prepared. To this mixture, 4 g of Epothilone B, as crystals, is added and stirred for approximately 30 minutes to provide the epothilone suspension. The Epothilone B suspension is passed through a 60 mesh screen to remove any agglomerates.

### Preparation of Coated Particles

The coated particles are prepared by applying the Epothilone B suspension onto base particles. The base particles are 18/20 mesh sugar beads, available as Sugar Spheres, NF particles, (Chr. Hansen, Inc., WI) having particle diameters in the range of greater than 0.85 mm and less than 1 mm.

The Epothilone B suspension is applied to the base particles by spraying using a fluid bed processor that is set up as a Wuster spray coating system. The spray coating system includes an Aeromatic-Fielder MP-MICRO™ fluid bed processor (Niro Inc., Maryland) equipped with a 0.8 mm spray tip. The fluid bed processor is charged with 90 g of the sugar beads and then preheated to approximately 50° C. for several minutes. The Epothilone B suspension is applied to the base particles with the following application and drying parameters: a spray rate of 1.1 g/minute with a spray atomization pressure of 1.8 bar (180 kilopascals), an inlet temperature of 68° C., an outlet temperature of 32° C., a product bed temperature of 32° C., and a fan speed of 4 m³/hr. During the application process, the Epothilone B suspension is slowly stirred.

After application of the Epothilone B suspension is completed, the inlet temperature is maintained at the final inlet temperature until the bed product temperature reaches 40° C.

The resulting coated particles contains 2.75 weight % of Epothilone B, based on the weight of the coated particle.

### Application of Subcoat Layer

A subcoat is applied to the coated particles. The subcoat solution is prepared by combining 5 g Opadry™ Clear Coat powder and 95 g water and stirring until a clear solution is obtained.

In the subcoating procedure, the fluid bed processor used to prepare the coated particles is employed. The fluid bed processor, which contained 80 g of the coated particles, is preheated to approximately 50° C. for several minutes. The subcoat layer is applied using the application and drying parameters disclosed hereinabove to the preparation of the coated particles. During the application process, the subcoated solution is slowly stirred. After application of the subcoat solution is completed, the inlet temperature is maintained at the final inlet temperature until the bed product temperature reached 40° C. The resulting subcoated coated particles contains approximately 3 weight % subcoat, based on the total weight of the subcoated coated particles.

### Application of Enteric Coating

An enteric coating is applied onto the coated particles having a subcoat. The enteric coating solution is prepared by first filtering Eudragit™ L30D55 polymer dispersion (Röhm GmbH and Co., Darmstadt, Germany) through a 60 mesh screen. Eudragit™ L30D55 polymer dispersion is an aqueous suspension containing methacrylic acid copolymer. The filtered Eudragit polymer dispersion (200 g) is diluted with 89.5 g water. Next, 9 g diethyl phthalate is added to the diluted Eudragit polymer dispersion, followed by the addition of 9.5 g of 1 N NaOH solution. The pH of the resulting enteric coating solution is 5.0±0.1.

In the enteric film coating procedure, the fluid bed processor used to prepare the coated particles is employed. The fluid bed processor, which contained 70 g of the subcoated coated particles, is preheated to approximately 50° C. for several minutes. The enteric coating solution is applied using the following application and drying parameters: 0.8 mm spray tip, 1.1 g/minute spray rate, spray atomization pressure was 1.8 bar, inlet temperature 65° C., outlet temperature 30° C., product bed temperature 30° C., and fan speed of 3.5 m³/hr. During the application process, the enteric coating solution is slowly stirred. After application of the enteric coating solution is completed, the inlet temperature is maintained at the final inlet temperature until the bed product temperature reaches 40° C. The resulting enteric coated beads have an average particle diameter of 1 mm.

Table 1 lists the composition of the enteric coated beads that are prepared in this example. The composition is reported as weight % of each ingredient based on the total weight of the enteric coated bead.

TABLE 1

| Ingredients | % w/w |
|---|---|
| A. Coated Particles | |
| Sugar spheres | 55.49 |
| Epothilone B | 1.60 |
| Binder (hydroxypropyl methylcellulose/PEG) | 1.00 |
| tris (hydroxymethyl) aminomethane | 0.10 |
| B. Subcoat Layer | |
| Subcoat (hydroxypropyl methylcellulose/PEG) | 1.80 |
| C. Enteric Coating | |
| methacrylic acid copolymer | 34.59 |
| Diethyl phthalate | 5.19 |
| NaOH | 0.22 |
| Total | 100.00 |

### Example 2

Enteric coated beads are prepared according to the general procedure described in Example 1, with the exception that the Epothilone D is substituted for Epothilone B.

What is claimed is:

1. An enteric coated bead comprising:
a) coated particle comprising:
i) a base particle; and
ii) an active ingredient layer disposed on said base particle, wherein said active ingredient layer comprises:
1) at least one epothilone or epothilone analog, or a pharmaceutically acceptable salt, solvate, clathrate, hydrate, or prodrug thereof; and
2) at least one binder; and
b) an enteric coating encapsulating said coated particle.

2. The enteric coated bead according to claim 1 comprising, based on weight of said enteric coated bead,
a) from about 10 to about 80 weight % of said base particle;
b) from about 0.1 to about 50 weight % of said active ingredient layer; and
c) from about 5 to about 55 weight % of said enteric coating.

3. The enteric coated bead according to claim 1, wherein said enteric coated bead is substantially free of moisture.

4. The enteric coated bead according to claim 1, wherein said at least one epothilone or epothilone analog is selected from compounds having the formula 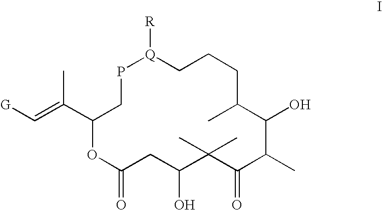
wherein Q is 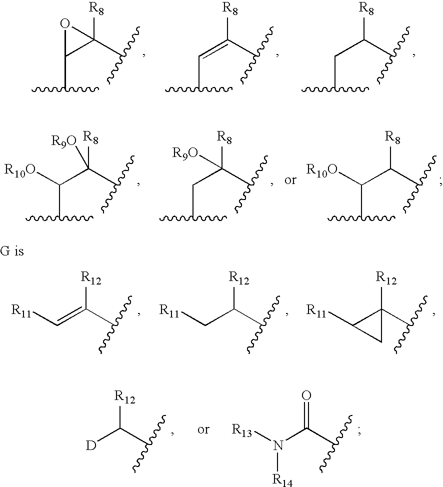
D is NR₂₈R₂₉, NR₃₀COR₃₁, or saturated heterocyclo;
R₁₃ and R₁₄ are independently H, alkyl, substituted alkyl, and/or aryl;
R₉, R₁₀, and R₃₁ are independently H, alkyl, and/or substituted alkyl;
R₈, R₁₁, R₁₂, R₂₈, R₃₀, R₃₂, and R₃₃ are independently H, alkyl, substituted alkyl, aryl, substituted aryl, cycloalkyl, and/or heterocyclo; and
R₂₉ is independently H, alkyl, substituted alkyl, aryl, substituted aryl, cycloalkyl, heterocyclo, R₃₂C═O, R₃₃SO₂, hydroxy, O-alkyl, or O-substituted alkyl; or a pharmaceutically acceptable salt, solvate, clathrate, hydrate, or prodrug thereof.

5. The enteric coated bead according to claim 1 wherein said at least one epothilone analog is [1S-[1R*,3R*(E),7R*,10S*,11R*,12R*,16S*]]-7,11-Dihydroxy-8,8,10,12,16-pentamethyl-3-[1-methyl-2-(2-aminomethyl-4-thiazolyl)ethenyl]-4,17-dioxabicyclo[14.1.0]heptadecane-5,9-dione; or a pharmaceutically acceptable salt, solvate, clathrate, hydrate, or prodrug thereof.

6. The enteric coated bead according to claim 1 wherein said at least one epothilone is epothilone A, epothilone B, epothilone C, epothilone D, epothilone E, or epothilone F; or a pharmaceutically acceptable salt, solvate, clathrate, hydrate, or prodrug thereof.

7. The enteric coated bead according to claim 6 comprising from about 0.1 to about 10 weight % of said subcoat layer, based on weight of said enteric coated bead.

8. The enteric coated bead according to claim 1 wherein said at least one binder is starch, gelatin, sucrose, glucose, dextrose, molasses, modified dextrins, lactose, acacia, sodium alginate, potassium alginate, methyl cellulose, hydroxymethyl cellulose, hydroxypropyl methylcellulose, hydroxypropyl cellulose, hydroxyethyl cellulose, guar gum, xanthan gum, polyvinylpyrrolidone, poly(vinylpyrrolidone-vinyl acetate) copolymers, or a mixture thereof.

9. The enteric coated bead according to claim 1 wherein said enteric coating comprises hydroxypropyl methylcellulose phthalate, polyvinyl acetate phthalate, cellulose acetate phthalate, or methacrylic acid copolymer.

10. The enteric coated bead according to claim 1 further comprising a subcoat layer interposed between said active ingredient layer and said enteric coating.

11. The enteric coated bead according to claim 10 wherein said subcoat layer is starch, gelatin, sucrose, glucose, dextrose, molasses, modified dextrins, lactose, acacia, sodium alginate, potassium alginate, methyl cellulose, hydroxymethyl cellulose, hydroxypropyl methylcellulose, hydroxypropyl cellulose, hydroxyethyl cellulose, guar gum, xanthan gum, polyvinylpyrrolidone, poly(vinylpyrrolidone-vinyl acetate) copolymers, or a mixture thereof.

12. A capsule comprising enteric coated beads according to claim 1.

13. The capsule according to claim 12 further comprising at least one hydrophobic material selected from talc, magnesium stearate, stearic acid, glyceryl behenate, hydrogenated cottonseed oil, trimyristin, triplamitan, tristearin, and fumed silica.

14. The capsule according to claim 12 wherein said at least one epothilone analog is [1S-[1R*,3R*(E),7R*,10S*,11R*,12R*,16S*]]-7,11-Dihydroxy-8,8,10,12,16-pentamethyl-3-[1-methyl-2-(2-aminomethyl-4-thiazolyl)ethenyl]-4,17-dioxabicyclo[14.1.0]heptadecane-5,9-dione; or a pharmaceutically acceptable salt, solvate, clathrate, hydrate, or prodrug thereof.

15. The capsule according to claim 12 wherein said at least one epothilone is epothilone A, epothilone B, epothilone C, epothilone D, epothilone E, or epothilone F; or a pharmaceutically acceptable salt, solvate, clathrate, hydrate, or prodrug thereof.

16. A process for preparing enteric coated beads, comprising:
a) providing base particles;
b) applying an active ingredient mixture and binder to said base particles, wherein said active ingredient mixture comprises:
i) at least one epothilone or epothilone analog, or a pharmaceutically acceptable salt, solvate, clathrate, hydrate, or prodrug thereof, and
ii) solvent, water, or a mixture thereof;
c) drying said base particles having application of said active ingredient mixture to provide coated particles; and
d) applying an enteric coating to said coated particles to provide said enteric coated beads.

17. The process according to claim 16 wherein said active ingredient mixture is an aqueous active ingredient suspension comprising particles of said at least one epothilone or epothilone analog in water.

18. The process according to claim 17 wherein said aqueous active ingredient suspension has a pH in the range of from about 6 to about 9.

19. The process according to claim 18 wherein said aqueous active ingredient suspension further comprises buffer.

20. The process according to claim 17 wherein said aqueous active ingredient suspension is applied to said base particles in step b) by spraying in a fluid bed spraying apparatus.

21. The process according to claim 17 wherein said base particles having application of said aqueous active ingredient suspension are dried in step c) at a temperature in the range of from about 25° C. to about 35° C. in said fluid bed spraying apparatus.

22. The process according to claim 16 wherein said base particles in step a) are substantially free of moisture.

23. The process according to claim 16 wherein said enteric coated beads are substantially free of moisture.

24. The process according to claim 16 wherein said at least one epothilone analog is [1S-[1R*,3R*(E),7R*,10S*,11R*,12R*,16S*]]-7,11-Dihydroxy-8,8,10,12,16-pentamethyl-3-[1-methyl-2-(2-aminomethyl-4-thiazolyl)ethenyl]-4,17-dioxabicyclo[14.1.0]heptadecane-5,9-dione, or a pharmaceutically acceptable salt, solvate, clathrate, hydrate, or prodrug thereof.

25. The process according to claim 16 wherein said at least one epothilone is epothilone A, epothilone B, epothilone C, epothilone D, epothilone E, or epothilone F, or a pharmaceutically acceptable salt, solvate, clathrate, hydrate, or prodrug thereof.

26. A method of treating cancer or other proliferative diseases in a mammal, comprising: administering orally an effective amount of at least one enteric coated bead of claim 1.

27. The method according to claim 26 wherein said mammal is human and said effective amount is in a range of from about 1 to about 500 mg/m² of said at least one epothilone or epothilone analog, or a pharmaceutically acceptable salt, solvate, clathrate, hydrate, or prodrug thereof.

28. The method according to claim 26 wherein said cancer is breast cancer or lung cancer.

29. The method according to claim 26 wherein said at least one epothilone analog is [1S-[1R*,3R*(E),7R*,10S*,11R*,12R*,16S*]]-7,11-Dihydroxy-8,8,10,12,16-pentamethyl-3-[1-methyl-2-(2-aminomethyl-4-thiazolyl)ethenyl]-4,17-dioxabicyclo[14.1.0]heptadecane-5,9-dione, or a pharmaceutically acceptable salt, solvate, clathrate, hydrate, or prodrug thereof.

30. The method according to claim 26 wherein said at least one epothilone is epothilone A, epothilone B, epothilone C, epothilone D, epothilone E, or epothilone F; or a pharmaceutically acceptable salt, solvate, clathrate, hydrate, or prodrug thereof.

31. The method according to claim 26 comprising administering orally a capsule comprising a multitude of said enteric coated beads.
